(19) United States
(12) Patent Application Publication
(10) Pub. No.: US 2025/0243136 A1
(43) Pub. Date: Jul. 31, 2025

(54) OLEFIN/PARAFFIN SEPARATION USING RECTIFIED ETS-4

(71) Applicant: Chevron Phillips Chemical Company LP, The Woodlands, TX (US)

(72) Inventor: Corey W. Knight, Houston, TX (US)

(21) Appl. No.: 18/428,349

(22) Filed: Jan. 31, 2024

(51) Int. Cl. C07C 7/13; B01D 53/047; B01J 20/10; B01J 20/28; B01J 20/30; C10G 9/00

(52) U.S. Cl. CPC C07C 7/13; B01D 53/047; B01D 53/0476; B01J 20/10; B01J 20/28033; B01J 20/2808; B01J 20/3078; B01J 20/3085; C10G 9/00; B01D 2253/106; B01D 2253/308; B01D 2256/24; B01D 2257/7022; C10G 2400/20

(57) ABSTRACT

A method of separating an alkene from a gas comprising the alkene and an alkane having a same carbon content as the alkene. The method includes contacting the gas with a rectified titanium silicate to selectivity adsorb the alkene and/or size exclude the alkane from the pores of the rectified titanium silicate, and recovering a product having a higher concentration of the alkene than the gas. A system for carrying out the method is also provided.

### CROSS-REFERENCE TO RELATED APPLICATIONS

None.

### STATEMENT REGARDING FEDERALLY SPONSORED RESEARCH OR DEVELOPMENT

Not applicable.

### TECHNICAL FIELD

The present disclosure relates to systems and methods for separating olefins and paraffins; more specifically, the present disclosure relates to systems and methods for separating olefins and paraffins via the use of a rectified titanium silicate; still more specifically, the present disclosure relates to systems and methods for separating olefins and paraffins via the use of a rectified titanium silicate having a controlled pore size.

### BACKGROUND

A need exists for systems and methods for separating olefins and paraffins.

### SUMMARY

Herein disclosed is a method of separating an alkene from a gas comprising the alkene and an alkane having a same carbon content as the alkene, the method comprising: contacting the gas with a rectified titanium silicate to selectivity adsorb the alkene and/or size exclude the alkane from the pores of the rectified titanium silicate; and recovering a product having a higher concentration of the alkene than the gas.

Also disclosed herein is a method of recovering an alkene from a hydrocarbon liquid stream, the method comprising: thermally cracking the hydrocarbon liquid stream to form a gas comprising an alkene and an alkane having a same carbon content as the alkene; contacting the gas with a rectified titanium silicate to selectively adsorb the alkene and/or size exclude the alkane; and recovering a product having an alkene content greater than that of the gas.

Further disclosed herein is a method comprising: providing a rectified titanium silicate having a controlled pore size via calcination of a titanium silicate, wherein the pore size of the rectified titanium silicate is smaller than a pore size of the titanium silicate; separating an alkene from an alkane having a same carbon number as the alkene by contacting a gas comprising the alkene and the alkane with the rectified titanium silicate to selectivity adsorb the alkene and/or size exclude the alkane from the pores of the rectified titanium silicate; and recovering a product having a higher concentration of the alkene than the gas.

Also provided herein is a system for separating an alkene from a gas comprising the alkene and an alkane having a same carbon content as the alkene, the method comprising: an adsorber comprising a bed of rectified titanium silicate, wherein the adsorber is configured for contacting the gas with the rectified titanium silicate to selectivity adsorb the alkene and/or size exclude the alkane from the pores of the rectified titanium silicate, wherein the adsorber has an inlet for the gas, an outlet for a removal, during adsorption, of a nonadsorbed gas comprising a lower concentration of the alkene than the gas, and an outlet for removal, during desorption, of a product gas comprising a higher concentration of the alkene than the gas; or a membrane comprising rectified titanium silicate, wherein the membrane is configured such that, during contacting of the gas with the rectified titanium silicate, the alkene passes through pores of the rectified titanium silicate and across a plane of the membrane, to provide, downstream of the membrane, a product gas comprising a higher concentration of the alkene than the gas.

### BRIEF DESCRIPTION OF THE DRAWINGS

The detailed description will reference the drawings briefly described below, wherein like reference numerals represent like parts, unless otherwise indicated.

FIG. 1 is a flow diagram of a method according to embodiments of this disclosure;

FIG. 2 is a flow diagram of another method, according to embodiments of this disclosure;

FIG. 3 is a schematic of a system, according to embodiments of this disclosure;

FIG. 4 is a schematic of another system, according to embodiments of this disclosure;

FIG. 5 is a plot of pressure with time for the Example; and

FIG. 6 is a plot of product concentration (volume percent) with time for the Example.

### DETAILED DESCRIPTION

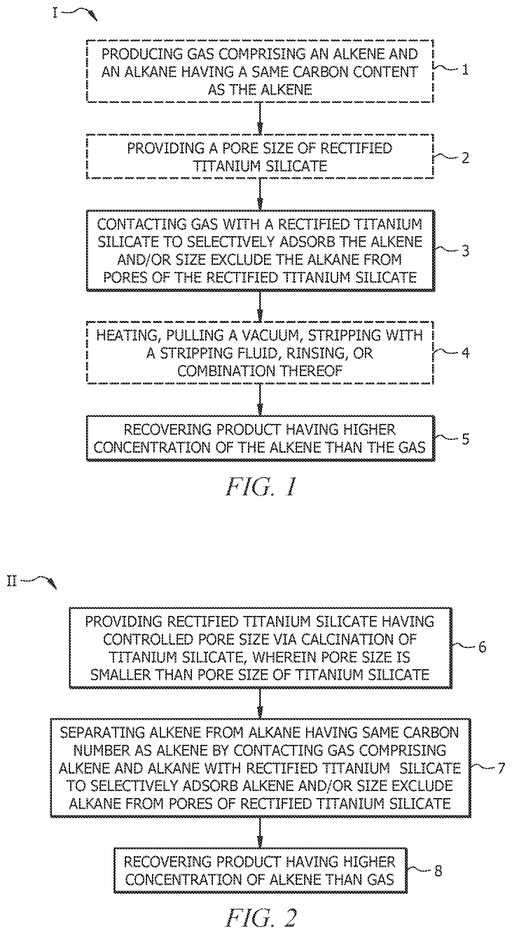

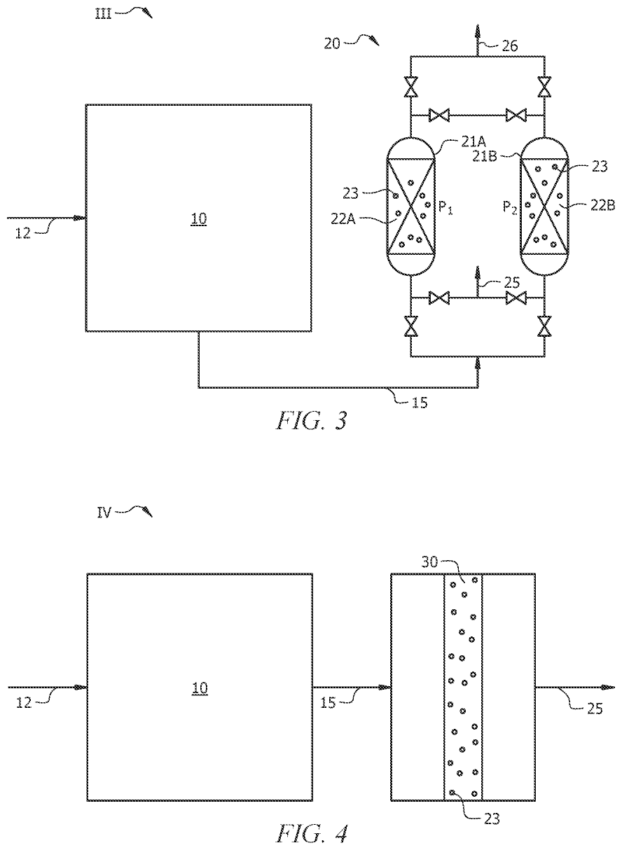

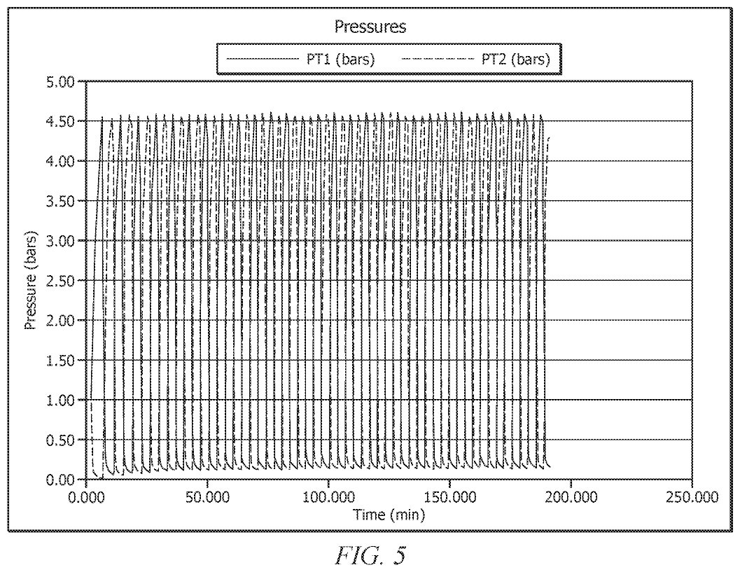

Herein disclosed is a method of separating an alkene (also referred to herein as an olefin) from a gas comprising the alkene and an alkane (also referred to as a paraffin) having a same carbon content as the alkene. The method will now be described with reference to FIG. 1, which is a flow diagram of a method I according to embodiments of this disclosure, FIG. 2, which is a flow diagram of another method II, according to embodiments of this disclosure, FIG. 3, which is a schematic of a system III, according to embodiments of this disclosure, and FIG. 4, which is a schematic of another system IV, according to embodiments of this disclosure. Method I comprises, as depicted at 3, contacting the gas 15 with a rectified titanium silicate 23 to selectivity adsorb the alkene and/or size exclude the alkane from the pores of the rectified titanium silicate; and, as depicted at 5, recovering a product 25 having a higher concentration of the alkene than the gas 15. Method I can further comprise, as depicted at 1, producing the gas comprising the alkene and the alkane having the same carbon content as the alkene, providing a desired pore size of rectified titanium silicate, as depicted at 2, and/or heating, pulling a vacuum, stripping, and/or rinsing the rectified titanium silicate 23, as depicted at 4. The details of the method will be provided hereinbelow.

ETS-4 is a microporous zeolite that contains titanium and silicate (e.g., a titanosilicate molecular sieve), first discovered by Engelhard Corporation. ETS-4 has a structure similar to that of inorganic microporous zeolites, and can comprise tetrahedral SiO₄ and octahedral TiO₆ units. ETS-4 can comprise five-coordinate titanium. The rectified titanium silicate 23 can comprise an ETS-4 titanosilicate (or another microporous zeolite containing titanium and silicate, encompassed herein by the term “ETS-4”) cross-exchanged with a cation selected from barium, strontium, calcium, hydrogen, or a combination thereof. For example, in embodiments, the rectified titanium silicate 23 contains a mixture of barium, strontium, hydrogen, and/or calcium cations. In embodiments, the rectified titanium silicate 23 comprises barium exchanged titanium silicate (Ba-ETS-4), which is a titanosilicate molecular sieve exchanged with barium, such that barium is present in the octahedral framework of the material. In embodiments, the rectified titanium silicate 23 comprises strontium exchanged titanium silicate (Sr-ETS-4), which is a titanosilicate molecular sieve exchanged with strontium such that strontium is present in the octahedral framework of the material. ETS-4 is synthesized with sodium (Na) cations (Na⁺), which are replaced with other cations, such as barium and hydrogen cations (Ba⁺⁺ and H⁺), according to this disclosure. Water molecules in the crystalline structure of ETS-4 are removed during the production of the rectified titanium silicate 23 as described herein (e.g., during the calcination described hereinbelow), thus allowing for contraction of the molecular framework of the rectified titanium silicate 23 and control (e.g., reduction) in the pore size of the rectified titanium silicate 23 relative to the starting titanium silicate (e.g., ETS-4) material. The rectified titanium silicate 23 can thus, in embodiments, have a calcination-controlled pore size. In an aspect, the ETS-4 titanosilicate is characterized by the formula [Na₉Si₁₂Ti₅O₃₈(OH)·₁₂H₂O]. In an aspect, the ETS-4 titanosilicate has the same structure as the zorite mineral, which has a framework of nenadkevichite-like chains, laterally connected by 4[SiO₄]/[TiO₆] units, corresponding to two different chemical environments for Si.

The rectified titanium silicate 23 can have a titania/silica mole ratio of from about 1.0 to about 10 from about 2 to about 9, or from about 3 to about 8. In embodiments, the pore size of the rectified titanium silicate 23 is in a range of from about 2 Å to about 4.5 Å, from about 2.5 Å to about 4 Å, or from about 2 Å to about 3.5 Å. The rectified titanium silicate 23 can be in the form of beads, pellets, spheres, or a combination thereof.

In embodiments, methods such as described, for example, in U.S. Pat. Nos. 4,853,202, 4,938,939, 6,395,067, 6,517,611, EP 0372132, EP 1042225, and/or WO 02/14219, the disclosure of each of which is hereby incorporated herein in its entirety for purposes not contrary to this disclosure, can be utilized in the formation of the rectified titanium silicate. In embodiments, as may be apparent to one of skill in the art and with the help of this disclosure, another method can be utilized to form the rectified titanium silicate.

As depicted at 2 of FIG. 1, in embodiments, the method of this disclosure can further comprise providing a pore size of the rectified titanium silicate 23. A desired pore size to effect a desired separation (e.g., ethane from ethylene, propane from propylene, butane from butylene, hexane from hexene, etc.) can be provided by forming the rectified titanium silicate 23 from ETS-4 as described hereinbelow. The rectified titanium silicate can be derived from an ETS-4 titanium silicate which has been cation exchanged and calcined at a temperature of from about 100° to about 300° C., from about 125° to about 275° C., or from about 150° to about 250° C. and subsequently cooled to adjust a pore size thereof. For example, in embodiments, the ETS-4 has been calcined at a temperature of about 180° C., 185° C., or 190° C. The calcination can be performed for at least 1, 5, 10, 15, or 20 hours.

Exchange with cations, such as barium, calcium, zinc, strontium, etc., as described herein can allow for fine tuning of the pore diameter of the titanium silicate adsorbent 23. Manipulating the pH of the cation (e.g., barium) exchange process can result in some of the cation (e.g., barium) being replaced with hydrogen which can limit the thermodynamic interaction between the cation (e.g., barium) and olefin (e.g., ethylene).

The rectified titanium silicate 23 can comprise titanium silicate (e.g., ETS-4) that has been subjected to: (1) a low temperature (125° C.-200° C.) annealing before the cation exchange; (2) techniques for the exhaustive exchange of the adsorbent (e.g., titanium silicate), such as column stripping; and (3) self-binding techniques to increase aggregate volumetric adsorbent (e.g., titanium silicate) concentration. The low temperature annealing at (1) can, without being limited by theory, provide a more uniform rectified titanium silicate 23, which can be activated to higher, better performing, temperatures. Exhaustive exchange of the titanium silicate adsorbent at (2) can, without being limited by theory, may instill disproportionate advantages to the titanium silicate adsorbent as complete exchange is approached. Self-binding techniques (3) can, without being limited by theory, increase effective bed adsorption capacity and/or efficiency.

Via this disclosure, titanium silicate (e.g., ETS-4) can be modified to both size-exclude paraffin (e.g., ethane) and weaken a bonding strength of alkene (e.g., ethylene) to the cation. That is, the pores of the rectified titanium silicate 23 can act like a molecular gate and the binding of the molecules to the cation inside the material can act like a sponge. For example, ETS-4 displays what is known as a ‘molecular gate’ effect, which refers to a shrinkage of the adsorbent pore diameter through dehydration at high temperatures. The rectified titanium silicate 23 can thus provide a combination of geometric and thermodynamic separation of olefins and paraffins.

As noted hereinabove, method I comprises contacting the gas 15 with the rectified titanium silicate 23, at 3, to selectivity adsorb the alkene and/or size exclude the alkane from the pores of the rectified titanium silicate 23, and recovering product 25 having a higher concentration of the alkene than the gas 15, at 5. The contacting at 3 and recovering at 5 can be effected by a pressure swing adsorption (PSA) process, a temperature swing adsorption process, or a vacuum swing adsorption process. In applications, a PSA process is utilized. In such instances, contacting the gas 15 with the rectified titanium silicate at 3 can comprise contacting the gas 15 with a particulate bed 22A/22B of the rectified titanium silicate 23, whereby the gas 15 flows over the particulate bed 22A/22B such that olefin (e.g., ethylene) is preferentially adsorbed, while the paraffin (e.g., ethane) passes through the particulate bed 22A/22B. Recovering the product 25 having the higher concentration of the alkene than the gas 15 can then be effected, at 5, by reducing a pressure of the particulate bed 22A/22B relative to a pressure of the particulate bed 22A/22B during the contacting 3. Once the particulate bed 22A/22B bed has reached its capacity for the olefin being adsorbed (e.g., ethylene), the particulate bed 22A/22B can be depressurized and the olefin desorbed from the particulate bed 22A/22B to leave as the product gas 25. Further removal of olefin (e.g., ethylene) from the particulate bed 22A/22B can be effected, as needed. For example, further desorption of olefin can be effected either by heating up the particulate bed 22A/22B, pulling a vacuum on the particulate bed 22A/22B, stripping of the particulate bed 22A/22B with another gas, such as, without limitation, steam or nitrogen, and/or rinsing the particulate bed 22A/22B. Accordingly, as depicted in FIG. 1, method I can further comprise, at 4, heating the particulate bed 22A/22B, pulling a vacuum on the particulate bed 22A/22B, stripping with a stripping fluid (e.g., steam, nitrogen), rinsing of the particulate bed 22A/22B with an alkane, alkene, hydrocarbon, water, nitrogen, oxygen, or a combination thereof to increase a recovery of the product 25.

The adsorption phase can occur at higher pressure than the regeneration or desorption phase which can, in embodiments, occur at or near atmospheric pressure or, if needed, under vacuum.

In embodiments, the method can include a rinse step in which a tail gas can be utilized to purge the particulate bed after the adsorption phase and before the depressurization. The rinse step can be employed to remove feed gas contamination and further concentrate the tail gas. In such embodiments, the tail gas can connect to a compressor that can pressurize the tail gas to be sent elsewhere and/or utilized in the rinse step.

As depicted in the embodiment of FIG. 1 and FIG. 2, the PSA unit 20 can comprise dual vessels 21A, 21B, such that one of the vessels can be in operation, while another of the dual vessels is being idle or being depressurized/regenerated. Each of the vessels 21A, 21B can comprise a particulate bed 22A/22B, respectively, of rectified titanium silicate. Accordingly, first vessel 21A can be in operation contacting the gas 15 comprising the alkene and the alkane with the rectified titanium silicate 23 of the particulate bed 22A, while second vessel 21B is being depressurized to remove adsorbed alkene 25 therefrom. During operation, the olefin can be adsorbed onto the rectified titanium silicate 23, and non-adsorbed gas 26 comprising the paraffin can be removed from PSA 20.

When the first vessel 21A needs regeneration, second vessel 21A can be put in operation for contacting the gas 15 comprising the alkene and the alkane with the rectified titanium silicate 23 of the particulate bed 22B of second vessel 21B, while first vessel 21A is being depressurized to remove adsorbed alkene 25 therefrom. In embodiments, the vessel in operation for adsorbing the olefin is operated at a pressure in a first pressure range. A vessel being regenerated can be operated at a pressure of less than the operating pressure of the vessel in operation to adsorb the alkene. For example, during regeneration, the operating pressure of the vessel being regenerated can be operated at a pressure in a second range. For example, if first vessel 21A has first vessel operating pressure P1, and vessel 21B has second vessel operating pressure P2, when first vessel 21A is in operation to adsorb the alkene while second vessel 21B is being regenerated to remove alkene 26 therefrom, first vessel operating pressure P1 of vessel 21A can be greater than second vessel operating pressure P2 of second vessel 21B. Conversely, when first vessel 21A is put into regeneration and second vessel 21B is being utilized to adsorb alkene, second vessel operating pressure P2 of second vessel 21B can be greater than first vessel operating pressure P1 of first vessel 21A.

Although two vessels 21A/21B have been discussed, the adsorber 20 (e.g., PSA 20) can include any number of (e.g., PSA) vessels, such as first vessel 21A and second vessel 21B. In embodiments, adsorber 20 (e.g., PSA) 20 is on a skid comprising 1, 2, 3, 4, or any number of PSA vessels 21. The vessels 21 can be operated as pairs, with one vessel of each pair being in operation while a second one of the pair is idling or being regenerated/depressurized.

As noted above, in embodiments, adsorber 20 is operated as a vacuum swing adsorber, and vessels 21A/21B are operated for vacuum swing adsorption of the olefin. As Sr-ETS-4 displays the same molecular gate effect as Ba-ETS-4 but has a stronger binding of olefin (e.g., ethylene) than Ba-ETS-4 and thus better selectivity, Sr-ETS-4 can be utilized, in embodiments, in a vacuum swing adsorption or steam stripping process, as a larger driving force may be needed to remove all the adsorbed olefin (e.g., ethylene).

The PSA can be operated at pressures, for example, between about 0.1 bara to about 50 bara. The PSA can include any number of beds, such as a 2 bed, 4 bed, 6 bed, PSA operation. In embodiments, the PSA can include rapid PSA, for example, having cycles of less than one minute.

As depicted in system IV of FIG. 4, in embodiments, the rectified titanium silicate 23 can be in the form of a membrane 30. In such applications, during the contacting of the gas 15 with the rectified titanium silicate 23, the alkene can pass through pores of the rectified titanium silicate 23 and across a plane of the membrane 30, and the product 25 can be obtained downstream of the membrane 30. Thus, in embodiments the rectified titanium silicate 23 is on a membrane 30 and the membrane 30 is utilized to achieve olefin/paraffin separation. Membrane 30 with the rectified titanium silicate allows for membrane separation of olefins and paraffins wherein a feed stream of gas 15 comprising both olefin and paraffin can be sent (e.g., to a skid with) one or more membranes 30. The olefin permeates through the membrane 30 with the filtrate, while the paraffin, that cannot pass through membrane 30, remains with the retentate. For example, in embodiments in which gas 15 comprises ethylene and ethane, ethylene can preferentially permeate through the membrane 30 based on the pore diameter of the rectified titanium silicate 23, whereas ethane will remain in the retentate due to size exclusion thereof from passing through the membrane 30.

In embodiments, the gas 15 further comprises acetylene. In such embodiments, the product 25 can comprise a higher concentration of acetylene than the gas 15.

The gas 15 comprising the alkene and the alkane having the same carbon number as the alkene can be any gas stream comprising an alkene and an alkane with the same carbon number. For example, and without limitation, the gas 15 can comprise ethylene and ethane, propylene and propane, butene and butane, hexene and hexane, and the like. Due to the herein described treatment of titanium silicate to provide a desired pore size, the system and method can be tailored for the separation of a variety of molecules, such as, for example, the separation of propane and molecules larger than propane (C3+) from ethylene and other molecules smaller than ethane, the separation of butane and molecules larger than butylene (C4+) from butylene and other molecules smaller than butane, etc.

As depicted in FIG. 1, a method of this disclosure can further include, at 1, producing the gas 15 comprising the alkane and the alkene having the same carbon number as the alkene. For example, with reference to system III of FIG. 3 and system IV of FIG. 4, the gas 15 can be produced, from a feedstream 12, via gas production apparatus 10. In embodiments, gas production apparatus 10 comprises an ethylene production plant, a polyethylene plant, a flex feed cracker, an ethane cracker, or a combination thereof. The gas 15 can be produced, at least in part, via the oxidative coupling of a feedstream 12 comprising methane, thermal cracking of a feedstream 12 comprising the alkane, and/or thermal cracking of a feedstream 12 comprising a hydrocarbon liquid ranging in boiling point from light straight-run gasoline to gas oil. In embodiments, the herein disclosed system and method can be utilized to separate olefins from corresponding (i.e., same carbon number) paraffins as an alternative to a conventional C2 splitter, C3 splitter, C4 splitter, C6 splitter, as an alternative olefin (e.g., ethylene) recovery unit, and/or to adsorb olefin during vent stream capture.

In embodiments, the method of this disclosure can be utilized for recovering an alkene from a hydrocarbon liquid stream (e.g., a feedstream 12 comprising a hydrocarbon liquid), and the method further includes, at 1, producing the gas 15 by thermally cracking the hydrocarbon liquid feedstream 12 to form the gas 15 comprising the alkene and the alkane having a same carbon content as the alkene. The gas produced via thermal cracking can comprise C3 hydrocarbons, and the method can further include separating C3 hydrocarbons (e.g., via distillation, or another rectified titanium silicate) to provide the gas 15 prior to the contacting of the gas 15 with the rectified titanium silicate 23 to separate ethylene from ethane.

With reference to FIG. 2, in embodiments, a method II of recovering an alkene from a hydrocarbon liquid stream, according to this disclosure, comprises, at 6, providing rectified titanium silicate 23 having a controlled pore size via calcination of titanium silicate, wherein the pore size of the rectified titanium silicate 23 is smaller than a pore size of the titanium silicate; at 7, separating an alkene from an alkane having a same carbon number as the alkene by contacting a gas 15 comprising the alkene and the alkane with the rectified titanium silicate 23 to selectivity adsorb the alkene and/or size exclude the alkane from the pores of the rectified titanium silicate 23; and, at 8, recovering a product 25 having a higher concentration of the alkene than the gas 15. As noted hereinabove, the rectified titanium silicate 23 can comprise an ETS-4 titanosilicate cross-exchanged with a cation selected from barium, strontium, calcium, hydrogen, or a combination thereof. As discussed hereinabove with reference to FIG. 1, contacting the gas 15 with the rectified titanium silicate 23 at 7 to selectivity adsorb the alkene and size exclude the alkane from the pores of the rectified titanium silicate 23 can comprise passing the gas to a particulate bed 22A/22B of the rectified titanium silicate 23. The contacting, at 7, and recovering, at 8, can be effected by a PSA process, as mentioned hereinabove, wherein recovering the product 25 having the higher concentration of the alkene than the gas at 8 can be effected by reducing a pressure of the particulate bed 22A/22B from the pressure of the particulate bed 22A/22B during the contacting.

Also as described hereinabove, the rectified titanium silicate 23 can be in the form of a membrane 30, and, in such embodiments, during the contacting of the gas 15 with the rectified titanium silicate 23, at 7, the alkene can pass through pores of the rectified titanium silicate 23 membrane 30 and across a plane of the membrane 30, and the product 25 can be obtained downstream of the membrane 30.

The rectified titanium silicate 23 can be a rectified titanium silicate 23 described and/or produced as described hereinabove. For example, the rectified titanium silicate 23 can comprise titanium silicate molecular sieve (e.g., ETS-4) ion exchanged with a cation selected from barium, strontium, calcium, hydrogen, or a combination thereof. The rectified titanium silicate 23 can have a titania/silica mole ratio of from about 1.0 to about 10.

The alkene and alkane in the gas 15 that are separated by the method II can be, for example, ethane and ethylene, propane and propylene, butane and butylene, or butane and butene.

As noted above with reference to method I of FIG. 1, method II can also further comprise controlling a pore size of the rectified titanium silicate 23 via calcination and subsequent cooling prior to the contacting. The pore size of the rectified titanium silicate 23 can be controlled during the formation of the rectified titanium silicate 23 to be in a range of from about 2.5 to about 4 Å.

Commercially, separation of olefins and paraffins has been effected primarily via cryogenic distillation. However, other techniques, such as using solid state copper, metal organic frameworks, zeolitic imidazolate frameworks, and other forms of adsorbents in a pressure swing process and membranes, for example, have been explored, but not become commercially widespread. The system and method of this disclosure provide for separation of olefins from paraffins via the use of a rectified titanium silicate 23.

Other advantages will be apparent to those of skill in the art and with the help of this disclosure.

### EXAMPLES

The embodiments having been generally described, the following examples are given as particular examples to demonstrate the practice and advantages of this disclosure. It is understood that the examples are given by way of illustration and are not intended to limit the specification or the claims in any manner.

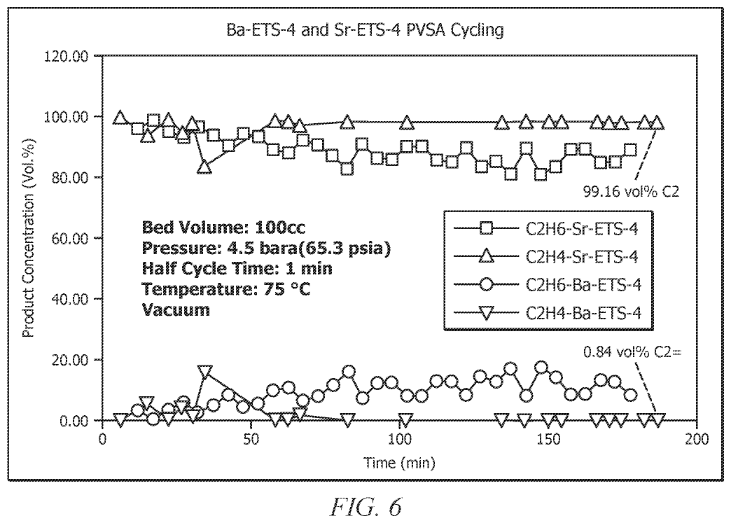

As depicted in FIG. 5, which is a plot of pressure with time, a 50/50 mixture of ethane and ethylene was fed to a lab PSA unit with Ba-ETS-4 and Sr-ETS-4 on 1 minute adsorption cycles and run for 190 minutes to allow the system to come to steady state. The Sr-ETS-4 showed initial excellent selectivity of ethylene over ethane, but showed more hysteresis and poorer performance than Ba-ETS-4. As depicted in FIG. 6, which is a plot of product concentration (volume percent) with time, a 99%+ recovery of ethylene was exhibited over about 100 cycles for Ba-ETS-4.

### Additional Description

The particular embodiments disclosed above are illustrative only, as the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present disclosure. Alternative embodiments that result from combining, integrating, and/or omitting features of the embodiment(s) are also within the scope of the disclosure. While compositions and methods are described in broader terms of “having”, “comprising,” “containing,” or “including” various components or steps, the compositions and methods can also “consist essentially of” or “consist of” the various components and steps. Use of the term “optionally” with respect to any element of a claim means that the element is required, or alternatively, the element is not required, both alternatives being within the scope of the claim.

Numbers and ranges disclosed above may vary by some amount. Whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent or other documents, the definitions that are consistent with this specification should be adopted.

The following are non-limiting, specific embodiments in accordance with the present disclosure:

In a first embodiment, a method of separating an alkene from a gas comprising the alkene and an alkane having a same carbon content as the alkene comprises: contacting the gas with a rectified titanium silicate to selectivity adsorb the alkene and/or size exclude the alkane from the pores of the rectified titanium silicate; and recovering a product having a higher concentration of the alkene than the gas.

A second embodiment can include the method of the first embodiment, wherein the rectified titanium silicate comprises an ETS-4 titanosilicate cross-exchanged with a cation selected from barium, strontium, calcium, hydrogen, or a combination thereof. In an aspect, the ETS-4 titanosilicate is characterized by the formula [Na₉Si₁₂Ti₅O₃₈(OH)·₁₂H₂O]. In an aspect, the ETS-4 titanosilicate has the same structure as the zorite mineral, which has a framework of nenadkevichite-like chains, laterally connected by 4[SiO₄]/[TiO₆] units, corresponding to two different chemical environments for Si.

A third embodiment can include the method of the second embodiment, wherein the rectified titanium silicate contains a mixture of cations.

A fourth embodiment can include the method of any one of the first to third embodiments, wherein the rectified titanium silicate is derived from an ETS-4 titanium silicate which has been calcined at a temperature of from about 100° to about 300° C., from about 125° to about 275° C., or from about 150° to about 250° C. and subsequently cooled to adjust a pore size thereof.

A fifth embodiment can include the method of the fourth embodiment, wherein the ETS-4 has been calcined at a temperature of about 170, 180, or 190° C., or in a range of from about 100° C. to about 300° C., from about 150° C. to about 250° C., or from about 200° C. to about 250° C. for at least 1, 5, 10, 15, or 20 hours.

A sixth embodiment can include the method of any one of the first to fifth embodiments, wherein contacting the gas with the rectified titanium silicate to selectivity adsorb the alkene and/or size exclude the alkane from the pores of the rectified titanium silicate comprise passing the gas to a particulate bed of the rectified titanium silicate.

A seventh embodiment can include the method of the sixth embodiment, wherein the contacting and recovering are effected by a pressure swing adsorption (PSA) process, wherein recovering the product having the higher concentration of the alkene than the gas is effected by reducing a pressure of the particulate bed relative to a pressure of the particulate bed during the contacting.

An eighth embodiment can include the method of the sixth or seventh embodiment, further comprising heating the particulate bed, pulling a vacuum on the particulate bed, stripping with a stripping fluid (e.g., steam, nitrogen), or a combination thereof to increase a recovery of the product.

A ninth embodiment can include the method of any one of the first to ninth embodiments, wherein the rectified titanium silicate is in the form of a membrane, and wherein, during the contacting the gas with the rectified titanium silicate, the alkene passes through pores of the rectified titanium silicate and across a plane of the membrane, and wherein the product is obtained downstream of the membrane.

A tenth embodiment can include the method of any one of the first to ninth embodiments, wherein the gas further comprises acetylene, and the product comprises a higher concentration of acetylene than the gas.

An eleventh embodiment can include the method of any one of the first to tenth embodiments, wherein the gas comprises a vent stream from a polyethylene or polypropylene production facility.

A twelfth embodiment can include the method of any one of the first to eleventh embodiments, wherein the gas is produced at least in part via the oxidative coupling of methane, thermal cracking of the alkane, and/or thermal cracking of a hydrocarbon liquid ranging in boiling point from light straight-run gasoline to gas oil.

A thirteenth embodiment can include the method of any one of the first to twelfth embodiments, wherein the alkene is ethylene and the alkane is ethane.

A fourteenth embodiment can include the method of any one of the first to thirteenth embodiments, wherein the alkene is propylene and the alkane is propane.

In a fifteenth embodiment, a method of recovering an alkene from a hydrocarbon liquid stream comprises: thermally cracking the hydrocarbon liquid stream to form a gas comprising an alkene and an alkane having a same carbon content as the alkene; contacting the gas with a rectified titanium silicate to selectively adsorb the alkene and/or size exclude the alkane; and recovering a product having an alkene content greater than that of the gas.

A sixteenth embodiment can include the method of the fifteenth embodiment, wherein the rectified titanium silicate comprises an ETS-4 titanosilicate cross-exchanged with a cation selected from barium, strontium, calcium, hydrogen, or a combination thereof.

A seventeenth embodiment can include the method of the fifteenth or sixteenth embodiment, wherein the gas comprises C3 hydrocarbons, and wherein the method further comprises separating C3 hydrocarbons (e.g., via distillation, or another rectified titanium silicate) prior to the contacting of the gas with the rectified titanium silicate.

An eighteenth embodiment can include the method of any one of the fifteenth to seventeenth embodiments, wherein contacting the gas with the rectified titanium silicate to selectivity adsorb the alkene and size exclude the alkane from the pores of the rectified titanium silicate comprises passing the gas to a particulate bed of the rectified titanium silicate.

A nineteenth embodiment can include the method of the eighteenth embodiment, wherein the contacting and recovering are effected by a pressure swing adsorption (PSA) process, and wherein recovering the product having the higher concentration of the alkene than the gas is effected by reducing a pressure of the particulate bed from the pressure of the particulate bed during the contacting.

A twentieth embodiment can include the method of the any one of the fifteenth to nineteenth embodiments, wherein the rectified titanium silicate is derived from an ETS-4 titanium silicate that has been calcined at a temperature in a range of from about 100° to about 300° C., from about 125° to about 275° C., or from about 150° to about 250° C.

A twenty first embodiment can include the method of any one of the fifteenth to twentieth embodiments, wherein the rectified titanium silicate is in the form of a membrane, and wherein, during the contacting the gas with the rectified titanium silicate, the alkene passes through pores of the rectified titanium silicate and across a plane of the membrane, and wherein the product is obtained downstream of the membrane.

A twenty second embodiment can include the method of the twenty first embodiment further comprising controlling a pore size of the rectified titanium silicate via calcination and subsequent cooling prior to the contacting.

A twenty third embodiment can include the method of any one of the fifteenth to twenty third embodiments, wherein the alkene is ethylene and the alkane is ethane.

A twenty fourth embodiment can include the method of any one of the fifteenth to twenty third embodiments, wherein the alkene is propylene and the alkane is propane.

In a twenty fifth embodiment, a method comprises: providing a rectified titanium silicate having a controlled pore size via calcination of a titanium silicate, wherein the pore size of the rectified titanium silicate is smaller than a pore size of the titanium silicate; separating an alkene from an alkane having a same carbon number as the alkene by contacting a gas comprising the alkene and the alkane with the rectified titanium silicate to selectivity adsorb the alkene and/or size exclude the alkane from the pores of the rectified titanium silicate; and recovering a product having a higher concentration of the alkene than the gas.

A twenty sixth embodiment can include the method of the twenty fifth embodiment, wherein the titanium silicate comprises titanium silicate molecular sieve (e.g., ETS-4) ion exchanged with a cation selected from barium, strontium, calcium, hydrogen, or a combination thereof, and having a titania/silica mole ratio of from about 1.0 to about 10.

A twenty seventh embodiment can include the method of the twenty fifth or twenty sixth embodiment, wherein the pore size of the rectified titanium silicate is in a range of from about 2.5 to about 4 Å.

In a twenty eighth embodiment, a system for separating an alkene from a gas comprising the alkene and an alkane having a same carbon content as the alkene comprises: an adsorber comprising a bed of rectified titanium silicate, wherein the adsorber is configured for contacting the gas with the rectified titanium silicate to selectivity adsorb the alkene and/or size exclude the alkane from the pores of the rectified titanium silicate, wherein the adsorber has an inlet for the gas, an outlet for a removal, during adsorption, of a nonadsorbed gas comprising a lower concentration of the alkene than the gas, and an outlet for removal, during desorption, of a product gas comprising a higher concentration of the alkene than the gas; or a membrane comprising rectified titanium silicate, wherein the membrane is configured such that, during contacting of the gas with the rectified titanium silicate, the alkene passes through pores of the rectified titanium silicate and across a plane of the membrane, to provide, downstream of the membrane, a product gas comprising a higher concentration of the alkene than the gas.

A twenty ninth embodiment can include the system of the twenty eighth embodiment, wherein the rectified titanium silicate comprises an ETS-4 titanosilicate cross-exchanged with a cation selected from barium, strontium, calcium, hydrogen, or a combination thereof.

A thirtieth embodiment can include the system of the twenty eighth or twenty ninth embodiment, wherein the rectified titanium silicate is derived from an ETS-4 titanium silicate which has been calcined at a temperature of from about 100° to about 300° C., from about 125° to about 275° C., or from about 150° to about 250° C. and subsequently cooled to adjust a pore size thereof.

A thirty first embodiment can include the system of the thirtieth embodiment, wherein the ETS-4 has been calcined at a temperature of about 170, 180, or 190° C., or in a range of from about 100° C. to about 300° C., from about 150° C. to about 250° C., or from about 200° C. to about 250° C. for at least 1, 5, 10, 15, or 20 hours.

A thirty second embodiment can include the system of any one of the twenty eighth to thirty first embodiments, wherein the adsorber comprises a pressure swing adsorber (PSA), a temperature swing adsorber, or a vacuum swing adsorber.

While preferred embodiments of the invention have been shown and described, modifications thereof can be made by one skilled in the art without departing from the teachings of this disclosure. The embodiments described herein are exemplary only, and are not intended to be limiting. Many variations and modifications of the invention disclosed herein are possible and are within the scope of the invention.

Numerous other modifications, equivalents, and alternatives, will become apparent to those skilled in the art once the above disclosure is fully appreciated. It is intended that the following claims be interpreted to embrace all such modifications, equivalents, and alternatives where applicable. Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

What is claimed is:

1. A method comprising:
contacting a gas comprising an alkene and an alkane having a same carbon content as the alkene with a rectified titanium silicate to selectivity adsorb the alkene and/or size exclude the alkane from the pores of the rectified titanium silicate; and
recovering a product having a higher concentration of the alkene than the gas.

2. The method of claim 1, wherein the rectified titanium silicate comprises an ETS-4 titanosilicate cross-exchanged with a cation selected from barium, strontium, calcium, hydrogen, or a combination thereof.

3. The method of claim 1, wherein the rectified titanium silicate is derived from an ETS-4 titanium silicate which has been calcined at a temperature of from about 100° to about 300° C., and subsequently cooled to adjust a pore size thereof.

4. The method of claim 1, wherein contacting the gas with the rectified titanium silicate to selectivity adsorb the alkene and/or size exclude the alkane from the pores of the rectified titanium silicate comprise passing the gas to a particulate bed of the rectified titanium silicate.

5. The method of claim 4, wherein the contacting and recovering are effected by a pressure swing adsorption (PSA) process, wherein recovering the product having the higher concentration of the alkene than the gas is effected by reducing a pressure of the particulate bed relative to a pressure of the particulate bed during the contacting.

6. The method of claim 4 further comprising heating the particulate bed, pulling a vacuum on the particulate bed, stripping with a stripping fluid, or a combination thereof to increase a recovery of the product.

7. The method of claim 1, wherein the rectified titanium silicate is in the form of a membrane, and wherein, during the contacting the gas with the rectified titanium silicate, the alkene passes through pores of the rectified titanium silicate and across a plane of the membrane, and wherein the product is obtained downstream of the membrane.

8. The method of claim 1, wherein the gas further comprises acetylene, and the product comprises a higher concentration of acetylene than the gas.

9. The method of claim 1, wherein the gas comprises a vent stream from a polyethylene or polypropylene production facility.

10. The method of claim 1, wherein the gas is produced at least in part via the oxidative coupling of methane, thermal cracking of the alkane, and/or thermal cracking of a hydrocarbon liquid ranging in boiling point from light straight-run gasoline to gas oil.

11. The method of claim 1, wherein the alkene is ethylene and the alkane is ethane.

12. The method of claim 1, wherein the alkene is propylene and the alkane is propane.

13. The method of claim 1 further comprising:
thermally cracking a hydrocarbon liquid stream to form the gas comprising the alkene and the alkane having the same carbon content as the alkene.

14. A method comprising:
providing a rectified titanium silicate having a controlled pore size via calcination of a titanium silicate, wherein the pore size of the rectified titanium silicate is smaller than a pore size of the titanium silicate;
separating an alkene from an alkane having a same carbon number as the alkene by contacting a gas comprising the alkene and the alkane with the rectified titanium silicate to selectivity adsorb the alkene and/or size exclude the alkane from the pores of the rectified titanium silicate; and
recovering a product having a higher concentration of the alkene than the gas.

15. The method of claim 14, wherein the titanium silicate comprises titanium silicate molecular sieve ion exchanged with a cation selected from barium, strontium, calcium, hydrogen, or a combination thereof, and having a titania/silica mole ratio of from about 1.0 to about 10.

16. The method of claim 14, wherein the pore size of the rectified titanium silicate is in a range of from about 2.5 to about 4 Å.

17. A system for separating an alkene from a gas comprising the alkene and an alkane having a same carbon content as the alkene, the method comprising:
an adsorber comprising a bed of rectified titanium silicate, wherein the adsorber is configured for contacting the gas with the rectified titanium silicate to selectivity adsorb the alkene and/or size exclude the alkane from the pores of the rectified titanium silicate, wherein the adsorber has an inlet for the gas, an outlet for a removal, during adsorption, of a nonadsorbed gas comprising a lower concentration of the alkene than the gas, and an outlet for removal, during desorption, of a product gas comprising a higher concentration of the alkene than the gas; or
a membrane comprising rectified titanium silicate, wherein the membrane is configured such that, during contacting of the gas with the rectified titanium silicate, the alkene passes through pores of the rectified titanium silicate and across a plane of the membrane, to provide, downstream of the membrane, a product gas comprising a higher concentration of the alkene than the gas.

18. The system of claim 17, wherein the rectified titanium silicate comprises an ETS-4 titanosilicate cross-exchanged with a cation selected from barium, strontium, calcium, hydrogen, or a combination thereof.

19. The system of claim 17, wherein the rectified titanium silicate is derived from an ETS-4 titanium silicate which has been calcined at a temperature of from about 100° to about 300° C., and subsequently cooled to adjust a pore size thereof.

20. The system of claim 17, wherein the adsorber comprises a pressure swing adsorber (PSA), a temperature swing adsorber, or a vacuum swing adsorber.
